(19) United States
(12) Patent Application Publication
(10) Pub. No.: US 2001/0053780 A1
(43) Pub. Date: Dec. 20, 2001

(54) Daily treatment for erectile dysfunction using a PDE5 inhibitor

(76) Inventors: John S. Whitaker, Woodinville, WA (US); Inigo Saenz de Tejada, Madrid (ES); Kenneth M. Ferguson, Bothell, WA (US)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(21) Appl. No.: 09/834,442

(22) Filed: Apr. 13, 2001

Related U.S. Application Data
(63) Continuation-in-part of application No. 09/558,911, filed on Apr. 26, 2000.
(60) Provisional application No. 60/132,036, filed on Apr. 30, 1999.

(51) Int. Cl. A61K 31/4985

(52) U.S. Cl. 514/250

(57) ABSTRACT

The present invention relates to phosphodiesterase (PDE) enzyme inhibitors and to their use in pharmaceutical articles of manufacture. In particular, the present invention relates to potent inhibitors of cyclic quanosine 3′,5′-monophosphate specific phosphodiesterase type 5 (PDE5) that when incorporated into a pharmaceutical product at about 1 to about 10 mg unit dosage are useful for the treatment of sexual dysfunction by daily administration of the PDE5 inhibitor. The articles of manufacture described herein are characterized by PDE5 inhibition, and accordingly, provide a benefit in therapeutic areas where inhibition of PDE5 is desired, especially erectile dysfunction, with minimization or elimination of adverse side effects resulting from inhibition of other phosphodiesterase enzymes and with an improvement of -vascular conditioning.

### CROSS REFERENCE TO RELATED APPLICATIONS

This application is a continuation-in-part of U.S. application Ser. No. 09/558,911, filed Apr. 26, 2000, which claims the benefit of provisional patent application Serial No. 60/132,036, filed Apr. 30, 1999.

### FIELD OF THE INVENTION

The present invention relates to phosphodiesterase (PDE) enzyme inhibitors and to their use in pharmaceutical articles of manufacture. In particular, the present invention relates to potent inhibitors of cyclic guanosine 3′,5′-monophosphate specific phosphodiesterase type 5 (PDE5) that when incorporated into a pharmaceutical product are useful for the treatment of sexual dysfunction.

### BACKGROUND OF THE INVENTION

The biochemical, physiological, and clinical effects of cyclic guanosine 3′,5′-monophosphate specific phosphodiesterase (cGMP-specific PDE) inhibitors suggest their utility in a variety of disease states in which modulation of smooth muscle, renal, hemostatic, inflammatory, and/or endocrine function is desired. Type 5 cGMP-specific phosphodiesterase (PDE5) is the major cGMP hydrolyzing enzyme in vascular smooth muscle, and its expression in penile corpus cavernosum has been reported (Taher et al., J. Urol., 149:285A (1993)). Thus, PDE5 is an attractive target in the treatment of sexual dysfunction (Murray, DN&P 6(3):150-56 (1993)).

A pharmaceutical product that provides a PDE5 inhibitor is currently available, and is marketed under the trademark VIAGRA®. The active ingredient in VIAGRA® is sildenafil. The product is sold as an article of manufacture including 25, 50, and 100 mg tablets of sildenafil and a package insert. The package insert provides that sildenafil is a more potent inhibitor of PDE5 than other known phosphodiesterases (greater than 80 fold for PDE1 inhibition, greater than 1,000 fold for PDE2, PDE3, and PDE4 inhibition). The IC₅₀ for sildenafil against PDE5 has been reported as 3 nM (Drugs of the Future, 22(2), pp. 128-143 (1997)), and as 3.9 nM (Boolell et al., Int. J. of Impotence Res., 8 p. 47-52 (1996)). Sildenafil is described as having a 4,000-fold selectivity for PDE5 versus PDE3, and only a 10-fold selectivity for PDE5 versus PDE6. Its relative lack of selectivity for PDE6 is theorized to be the basis for abnormalities related to color vision

While sildenafil has obtained significant commercial success, problems in the treatment of erectile dysfunction (ED) still exist. First, ED therapy using sildenafil is based on an on-demand or PRN therapy. “On demand” dosing is defined as an acute administration of a drug for treating erectile dysfunction prior to expected sexual activity. The user therefore must plan ahead, and, as presently labeled, ingest a relatively large oral dose (i.e., at least 25 mg) of sildenafil at least one hour prior to engaging in sexual activity. The onset of beneficial effects may be delayed when sildenafil is administered with a meal.

Second, the relatively large on-demand dose of sildenafil results in significant adverse side effects, including facial flushing (10% incidence rate). Thus, even with the availability of sildenafil, there remains a need to identify improved pharmaceutical products that are useful and more convenient in treating sexual dysfunction.

The present invention provides an article of manufacture for human pharmaceutical use, comprising a package insert, a container, and an oral dosage form comprising a PDE5 inhibitor at unit dosages between about 1 and about 10 mg/dosage form. The package insert provides a dosing regimen characterized by a chronic administration of the PDE5 inhibitor. The beneficial effects of a chronic dosing regimen were observed in clinical studies and through the discovery that the administration of a PDE5 inhibitor improves or conditions the vasculature such that the corpus cavernosum smooth muscle tissue responds to therapy at doses below that required to yield the same response with on-demand or acute therapy. The benefits of a low, chronic administration of a PDE5 inhibitor include improved vascular response to cGMP-stimulated relaxation in the corpus cavernosum smooth muscle tissue, lower toxicity attributed to a lower dose of PDE5 inhibitor, and a return to normalcy, i.e., the patient is not required to plan sexual activity around administration of the PDE5 inhibitor. The dosing regimen of the present invention allows a spontaneity of sexual activity desired by the patient.

### SUMMARY OF THE INVENTION

The present invention provides an article of manufacture for human pharmaceutical use, comprising a package insert, a container, and an oral dosage form comprising about 1 to about 10 mg of a PDE5 inhibitor per dosage form for chronic, and preferably daily, dosing.

The present invention further provides a method of treating male erectile dysfunction comprising administering to a patient in need thereof an oral dosage form containing about 1 to about 10 mg of a PDE5 inhibitor, chronically, up to a total dose of 10 mg/day.

The present invention further provides a method of improving the relaxant response in corpus cavernosum smooth muscle tissue, which comprises chronically administering a dose of 1 mg/day to 10 mg/day of a PDE5 inhibitor.

The present invention provides an article of manufacture for human pharmaceutical use, comprising a package insert, a container, and an oral dosage form comprising about 1 to about 10 mg of a selective PDE5 inhibitor, said package insert providing for a chronic administration of the PDE5 inhibitor to treat a patient suffering from erectile dysfunction.

The present invention provides an article of manufacture for human pharmaceutical use, comprising a package insert, a container, and an oral dosage form of a selective PDE5 inhibitor; said package insert providing for a chronic administration of the PDE5 inhibitor to treat a patient suffering from erectile dysfunction.

The present invention further provides an article of manufacture for human pharmaceutical use comprising:

(a) an oral dosage form comprising about 1 to about 10 mg of a PDE5 inhibitor having an IC₅₀ less than 10 nM, and a sufficient bioavailability to be effective in about 1 to about 10 mg unit oral dosages;

(b) a package insert providing that the PDE5 inhibitor is useful to treat sexual dysfunction in a patient in need thereof, and has a chronic dosing regimen of about 1 to about 10 mg/day, wherein the chronic dosing regimen improves vascular conditioning; and

(c) a container.

The present invention further provides an article of manufacture for human pharmaceutical use comprising:

(a) an oral dosage form comprising about 1 to about 10 mg of a PDE5 inhibitor having

(i) an IC₅₀ less than 10 nM, and

(ii) a sufficient bioavailability to be effective in about 1 to about 10 mg unit oral dosages;

(b) a package insert providing that the PDE5 inhibitor is useful to treat sexual dysfunction in a patient in need thereof, and has a chronic dosing regimen of about 1 to about 10 mg/day, wherein the chronic dosing regimen improves vascular conditioning; and

(c) a container.

### DETAILED DESCRIPTION

For purposes of the present invention as disclosed and described herein, the following terms and abbreviations are defined as follows.

The term “container” means any receptacle and closure therefor suitable for storing, shipping, dispensing, and/or handling a pharmaceutical product.

The term “IC₅₀” is the measure of potency of a compound to inhibit a particular PDE enzyme (e.g., PDE1c, PDE5, or PDE6). The IC₅₀ is the concentration of a compound that results in 50% enzyme inhibition in a single dose-response experiment. Determining the IC₅₀ value for a compound is readily carried out by a known in vitro methodology generally described in Y. Cheng et al., Biochem. Pharmacol., 22, pp. 3099-3108 (1973).

The term “package insert” means information accompanying the product that provides a description of how to administer the product, along with the safety and efficacy data required to allow the physician, pharmacist, and patient to make an informed decision regarding use of the product. The package insert generally is regarded as the “label” for a pharmaceutical product.

The term “oral dosage form” is used in a general sense to reference pharmaceutical products administered orally. Oral dosage forms are recognized by those skilled in the art to include such forms as liquid formulations, tablets, capsules, and gelcaps.

The terms “day” and “daily” refer to the administration of the product one or more times, generally one to three times, still more preferably one time, per about 24-hour period. “About 24-hour period” refers to a time span of about 20 to about 28 hours.

The term “chronic or chronically” refers to the regular administration of the product in intervals unrelated to the onset of sexual activity. To receive the full benefit of the present invention, chronic administration generally refers to regular administration for an extended period, preferably daily for three or more days, and still more preferably daily as long as the patient suffers from erectile dysfunction (in the absence of therapy). The term “chronic” administration encompasses other regimens in addition to daily dosing. For example, chronic administration encompasses administration of a sustained release formulation that provides sufficient PDE5 inhibitor on a regular basis and unrelated to the onset of sexual activity. Contrary to acute or on-demand administration, chronic administration does not link the administration of the PDE5 inhibitor to the onset of sexual activity (e.g., one hour prior to intercourse).

The term “PDE5 inhibitor” refers to compounds having an IC₅₀ value for inhibition of PDE5 of less than 10 nM. Preferred PDE5 inhibitors are selective for PDE5 inhibition, such as those having:

(1) an IC₅₀ value for the inhibition of PDE5 at least 100 times less than the IC₅₀ value for the inhibition of PDE6;

(2) an IC₅₀ value for the inhibition of PDE5 at least 1,000 times less than the IC₅₀ value for the inhibition of PDE1c; and

(3) an IC₅₀ value for the inhibition of PDE5 less than 10 nM.

PDE5 inhibitors vary significantly in chemical structure, and their use in the present invention is not dependent on chemical structure, but rather on the potency parameters disclosed herein.

The term “vision abnormalities” means abnormal vision characterized by blue-green vision believed to be caused by PDE6 inhibition.

The term “free drug” means solid particles of drug not intimately embedded in a polymeric co-precipitate.

As previously stated, the present invention is directed to an article of manufacture for human pharmaceutical use, comprising a package insert, a container, and a dosage form comprising about 1 to about 10 mg of a PDE5 inhibitor per unit dosage form. A PDE5 inhibitor useful in the present invention is a PDE5 inhibitor having an IC₅₀ value for PDE5 inhibition of less than 10 nM, and is sufficiently bioavailable to be effective in about 1 to about 10 mg unit dosages.

Preferred PDE5 inhibitors selectively inhibit PDE5 versus PDE6 and PDE1c. Selectivity is quantified by the differential in IC₅₀. The differential is expressed as a PDE6/PDE5 ratio of IC₅₀ values, i.e., the ratio of the IC₅₀ value versus PDE6 to the IC₅₀ value versus PDE5 (PDE6/PDE5) is greater than 100, more preferably greater than 300, and most preferably greater than 500.

Similarly, the ratio of IC₅₀ value versus PDE1c to IC₅₀ value versus PDE5 (PDE1c/PDE5) is greater than 1000. Preferred PDE5 inhibitors have a greater than 3,000 fold differential between the inhibition of PDE5 and PDE1c, more preferably greater than a 5,000 fold differential between IC₅₀ value versus PDE5 and PDE1c. The potency of the inhibitor, as represented by the IC₅₀ value versus PDE5, is less than 10 nM, preferably less than 5 nM, more preferably less than 2 nM, and most preferably less than 1 nM.

The package insert provides a description of how to administer a pharmaceutical product, along with the safety and efficacy data required to allow the physician, pharmacist, and patient to make an informed decision regarding the use of the product. The package insert generally is regarded as the label of the pharmaceutical product. The package insert incorporated into the present article of manufacture indicates that the PDE5 inhibitor is useful in the treatment of conditions wherein inhibition of PDE5 is desired, particularly sexual dysfunction, and particularly male erectile dysfunction and female sexual arousal disorder.

The package insert also provides instructions to administer one or more about 1 to about 10 mg unit dosage forms, chronically, and preferably daily, for at least three days, up to a maximum total dose of 10 mg per day. The dose administered typically is about 1 to about 10 mg/day, more preferably about 2 to about 10 mg, and most preferably an about 5 mg to about 10 mg dosage form administered daily.

Because a presently claimed article of manufacture provides a chronic dosing regimen that is more efficacious than the equivalent on-demand or acute dose, incidences of side effects are notably reduced. Therefore, the preferred article of manufacture provides a package insert having reported incidences of flushing below 2%, preferably below 1%, and most preferably below 0.5%, of the patients administered the dosage form. The incidence rate of flushing demonstrates marked improvement over prior pharmaceutical products containing a PDE5 inhibitor.

The container used in the present article of manufacture is conventional in the pharmaceutical arts. Generally, the container is a blister pack, foil packet, glass or plastic bottle and accompanying cap or closure, or other such article suitable for use by the patient or pharmacist. Preferably, the container is sized to accommodate 1-1000 solid dosage forms, preferably 1 to 500 solid dosage forms, and most preferably, 5 to 30 solid dosage forms.

Oral dosage forms are recognized by those skilled in the art to include, for example, such forms as liquid formulations, tablets, capsules, and gelcaps. Preferably the dosage forms are solid dosage forms, particularly, tablets comprising about 1 to about 10 mg of a PDE5 inhibitor. Any pharmaceutically acceptable excipients for oral use are suitable for preparation of such dosage forms. Suitable pharmaceutical dosage forms include coprecipitate forms described, for example, in Butler U.S. Pat. No. 5,985,326, incorporated herein by reference. In preferred embodiments, the unit dosage form of the present invention is a solid free of a coprecipitate form of the PDE5 inhibitor, but rather contains a solid PDE5 inhibitor as a free drug.

Preferably, the tablets comprise pharmaceutical excipients generally recognized as safe such as lactose, microcrystalline cellulose, starch, calcium carbonate, magnesium stearate, stearic acid, talc, and colloidal silicon dioxide, and are prepared by standard pharmaceutical manufacturing techniques as described in Remington's Pharmaceutical Sciences, 18th Ed., Mack Publishing Co., Easton, Pa. (1990). Such techniques include, for example, wet granulation followed by drying, milling, and compression into tablets with or without film coating; dry granulation followed by milling, compression into tablets with or without film coating; dry blending followed by compression into tablets, with or without film coating; molded tablets; wet granulation, dried and filled into gelatin capsules; dry blend filled into gelatin capsules; or suspension and solution filled into gelatin capsules. Generally, the solid dosage forms have identifying marks which are debossed or imprinted on the surface.

The oral dosage form also can be in the form of sustained release formulation that chronically provides about 1 to about 10 mg/day of the PDE5 inhibitor to an individual over the course of a few to several days.

The present invention is based on detailed experiments and clinical trials, and the unexpected observations that sexual dysfunction can be treated using a chronic, low dose of a PDE5 inhibitor having an IC₅₀ value for inhibition of PDE5 less than 10 nM.

A chronic, and preferably daily, dosing regimen of about 1 to about 10 mg of a PDE5 inhibitor also provides other benefits including (a) spontaneity in sexual relations, (b) unexpected efficacy for such a low oral dose of PDE5 inhibitor, including an observation of a greater response to the PDE5 inhibitor from a lower chronic PDE5 inhibitor dose than to the currently labeled 25 mg acute, on-demand dose of sildenafil, and (c) no to low adverse effects attributed to the selective PDE5 inhibitor and a low dose.

Overall, it has been demonstrated that chronic dosing of a PDE5 inhibitor having the properties enumerated above provides the same ox improved efficacy at about 1 mg to 10 mg than a higher acute on-demand dosage presently administered. The enhanced efficacy demonstrated by low daily dosing of a PDE5 inhibitor in treating erectile dysfunction is not dependent on drug accumulation, but rather results from improved vascular responsiveness when the PDE5 inhibitor is present continuously, or essentially continuously, in plasma.

The “vascular conditioning” effect has not been demonstrated previously with PDE5 inhibitors in particular, or PDE inhibitors in general. In particular, vascular conditioning has not been observed in on-demand dosing of a PDE5 inhibitor, or in individuals taking an acute PDE5 inhibitor dose for a short time span of two to three days. It is expected that vascular conditioning occurs after chronic administration of the PDE5 inhibitor, for example, after about three daily doses of up to 10 mg, preferably after five days of daily dosing, and more preferably after seven days of daily dosing. In addition, after about three days of daily dosing, intermittently missing one chronic dose may lead to a reduction in vascular conditioning, but not a complete loss of conditioning.

It is theorized, but not relied upon herein, that vascular conditioning is caused by a partial or complete reversal of circulatory dysfunctions in penile circulation arising from conditions such as diabetes, atherosclerosis, smoking, hypertension, or a combination of such factors. These conditions result in thickening of the arterial wall, decreased arterial compliance, and decreased responsiveness to endogenous vasodilators, such as nitric oxide.

PDE5 inhibitors vary significantly in chemical structure, and the use of a PDE5 inhibitor as defined in the present invention is not dependent on a particular chemical structure, but rather on the critical parameters outlined herein. However, preferred compounds having the required potency and preferred selectivity can be readily identified by tests described herein from compounds described in Daugan U.S. Pat. No. 5,859,006, Daugan et al. U.S. Pat. No. 5,981,527, and Daugan et al. U.S. Pat. No. 6,001,847, each of which is incorporated herein by reference.

Preferred compounds of Daugan U.S. Pat. No. 5,859,006 and Daugan et al. U.S. Pat. No. 5,981,527 are represented by structural formula (I):

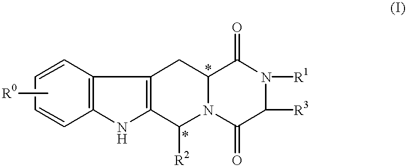

wherein R⁰ is selected from the group consisting of hydrogen, halogen, and C₁₋₆alkyl;

R¹ is selected from the group consisting of hydrogen, C₁₋₆alkyl, C₂₋₆alkenyl, C₂₋₆alkynyl, haloC₁₋₆-alkyl, C₃₋₈cycloalkyl, C₃₋₈cycloalkylC₁₋₃alkyl, aryl-C₁₋₃alkyl, wherein aryl is phenyl or phenyl substituted with one to three substituents selected from the group consisting of halogen, C₁₋₆alkyl, C₁₋₆alkoxy, methylenedioxy, and mixtures thereof, and hetero-arylC₁₋₃alkyl, wherein heteroaryl is thienyl, furyl, or pyridyl, each optionally substituted with one to three substituents selected from the group consisting of halogen, C₁₋₆alkyl, C₁₋₆alkoxy, and mixtures thereof;

R² represents an optionally substituted monocyclic aromatic ring selected from benzene, thiophene, furan, and pyridine, or an optionally substituted bicyclic ring

[chemical structure]

attached to the rest of the molecule via one of the benzene ring carbon atoms and wherein the fused ring A is a 5- or 6-membered ring, saturated or partially or fully unsaturated, and comprises carbon atoms and optionally one or two heteroatoms selected from the group consisting of oxygen, sulphur and nitrogen;

R³ represents hydrogen or C₁₋₃alkyl, or R¹ and R³ together represent a 3- or 4-membered alkyl or alkenyl chain; and salts and solvates thereof.

Other preferred compounds are those of formula (I) wherein:

R⁰ is hydrogen, halogen, or C₁₋₆alkyl;

R¹ is hydrogen or C₁₋₆alkyl;

R² is the bicyclic ring

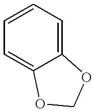

which can be optionally substituted by one or more groups selected from halogen and C₁₋₃alkyl; and

R³ is hydrogen or C₁₋₃alkyl.

Preferred compounds are:

(6R,12aR)-2,3,6,7,12,12a-hexahydro-2-methyl-6-(3,4-methylenedioxyphenyl)pyrazino[2′,1′:6,1]pyrido[3,4-b]indole-1,4-dione; and

(3S,6R,12aR)-2,3,6,7,12,12a-hexahydro-2,3-dimethyl-6-(3,4-methylenedioxyphenyl)pyrazino[2′,1′:6,1]-pyrido[3,4-b]indole-1,4-dione; and physiologically acceptable salts and solvates (e.g., hydrates) thereof.

An especially preferred selective PDE5 inhibitor useful in the present invention is (6R-trans)-6-(1,3-benzodioxol-5-yl)-2,3,6,7,12,12a-hexahydro-2-methylpyrazino[1′,2′:1,6]pyrido[3,4-b]indole-1,4-dione, alternatively named (6R,12aR) -2,3,6,7,12,12a-hexahydro-2-methyl-6-(3,4-methylene-dioxyphenyl)pyrazino[2′,1′:6,1]pyrido[3,4-b]indole-1,4-dione, which is disclosed in Daugan U.S. Pat. No. 5,859,006, and represented by structural formula (II):

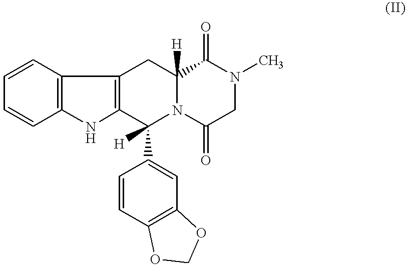

Other exemplary compounds useful in the present invention are disclosed in Daugan et al. U.S. Pat. No. 6,001,847, WO 97/43287, and WO 00/15639, incorporated herein by reference.

In addition, sildenafil and vardenafil can be used as the PDE5 inhibitor for daily dosing.

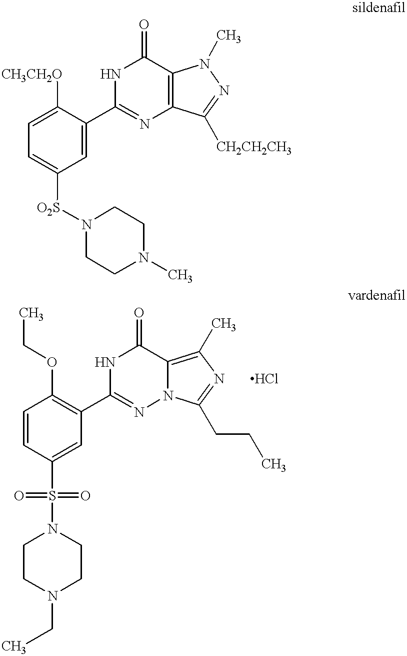

With respect to sildenafil and vardenafil, the dose for chronic administration is about 1 to about 25 mg/day, and preferably about 1 to about 20 mg/day.

Other useful PDE5 inhibitors that can be used in a chronic dosing regimen of the present invention include, but are not limited to:

5-(2-ethoxy-5-morpholinoacetylphenyl)-1-methyl-3-n-propyl-1,6-dihydro-7H-pyrazolo[4,3-d]pyrimidin-7-one;

5-(5-morpholinoacetyl-2-n-propoxyphenyl)-1-methyl-3-n-propyl-1,6-dihydro-7H-pyrazolo[4,3-d]pyrimidin-7-one;

5-[2-allyloxy-5-(4-methyl-1-piperazinylsulphonyl)-phenyl]-1-methyl-3-n-propyl-1,6-dihydro-7H-pyrazolo-[4,3-d]pyrimidin-7-one;

5-{2-ethoxy-5-[4-(2-propyl)-1-piperazinylsulphonyl]-phenyl}-1-methyl-3-n-propyl-1,6-dihydro-7H-pyrazolo-[4,3-d]pyrimidin-7-one;

5-{2-ethoxy-5-[4-(2-hydroxyethyl)-1-piperazinylsulphonyl)phenyl}-1-methyl-3-n-propyl-1,6-dihydro-7H-pyrazolo [4,3-d]pyrimidin-7-one;

5-{5-[4-(2-hydroxyethyl)-1-piperazinylsulphonyl]-2-n-propoxyphenyl}-1-methyl-3-n-propyl-1,6-dihydro-7H-pyrazolo[4,3-d]pyrimidin-7-one;

5-[2-ethoxy-5-(4-methyl-1-piperazinylcarbonyl)-phenyl]-1-methyl-3-n-propyl-1,6-dihydro-7H-pyrazolo-[4,3-d]pyrimidin-7-one; and

5-[2-ethoxy-5-(1-methyl-2-imidazolyl)phenyl]-1-methyl-3-n-propyl-1,6-dihydro-7H-pyrazolo[4,3-d]pyrimidin-7-one.

### PREPARATIONS

### Human PDE5 Preparation

Recombinant production of human PDE5 was carried out essentially as described in Example 7 of U.S. Pat. No. 5,702,936, incorporated herein by reference, except that the yeast transformation vector employed, which is derived from the basic ADH2 plasmid described in V. Price et al. Methods in Enzymology, 1985, pages 308-318 (1990), incorporated yeast ADH2 promoter and terminator sequences rather than ADH1 promoter and terminator sequences and the Saccharomyces cerevisiase host was the protease-deficient strain BJ2-54 deposited on Aug. 31, 1998 with the American Type Culture Collection, Manassas, Va., under accession number ATCC 74465. Transformed host cells were grown in 2×SC-leu medium, pH 6.2, with trace metals, and vitamins. After 24 hours, YEP medium containing glycerol was added to a final concentration of 2×YEP/3% glycerol. Approximately 24 hours later, cells were harvested, washed, and stored at −70° C.

Cell pellets (29 g) were thawed on ice with an equal volume of lysis buffer (25 mM Tris-Cl, pH 8, 5 mM MgCl₂, 0.25 mM dithiothreitol, 1 mM benzamidine, and 10 μM ZnSO₄). Cells were lysed in a microfluidizer with N₂ at 20,000 psi. The lysate was centrifuged and filtered through 0.45 μm disposable filters. The filtrate was applied to a 150 mL column of Q Sepharose Fast Flow (Pharmacia). The column was washed with 1.5 volumes of Buffer A (20 mM Bis-Tris Propane, pH 6.8, 1 mM MgCl₂, 0.25 mM dithiothreitol, 10 μM ZnSO₄) and eluted with a step gradient of 125 mM NaCl in Buffer A followed by a linear gradient of 125-1000 mM NaCl in Buffer A.

Active fractions from the linear gradient were applied to a 180 mL hydroxyapatite column in Buffer B (20 mM Bis-Tris Propane (pH 6.8), 1 mM MgCl₂, 0.25 mM dithiothreitol, 10 μM ZnSO₄, and 250 mM KCl). After loading, the column was washed with 2 volumes of Buffer B and eluted with a linear gradient of 0-125 mM potassium phosphate in Buffer B. Active fractions were pooled, precipitated with 60% ammonium sulfate, and resuspended in Buffer C (20 mM Bis-Tris Propane, pH 6.8, 125 mM NaCl, 0.5 mM dithiothreitol, and 10 μM ZnSO₄). The pool was applied to a 140 mL column of Sephacryl S-300 HR and eluted with Buffer C. Active fractions were diluted to 50% glycerol and stored at −20° C. The resultant preparations were about 85% pure by SDS-PAGE.

### Assay for PDE Activity

Activity of PDE5 can be measured by standard assays in the art. For example, specific activity of any PDE can be determined as follows. PDE assays utilizing a charcoal separation technique were performed essentially as described in Loughney et al., (1996), The Journal of Biological Chemistry, 271:796-806. In this assay, PDE5 activity converts [³²P] cGMP to [³²P] 5′GMP in proportion to the amount of PDE5 activity present. The [³²P]5′GMP then is quantitatively converted to free [32P] phosphate and unlabeled adenosine by the action of snake venom 5′-nucleotidase. Hence, the amount of [³²P] phosphate liberated is proportional to enzyme activity. The assay is performed at 30 C. in a 100 μL reaction mixture containing (final concentrations) 40 mM Tris-Cl (pH 8.0), 1 μM ZnSO₄, 5 mM MgCl₂, and 0.1 mg/mL bovine serum albumin. PDE5 is present in quantities that yield <30% total hydrolysis of substrate (linear assay conditions). The assay is initiated by addition of substrate (1 mM [³²P] cGMP), and the mixture is incubated for 12 minutes. Seventy-five (75) μg of Crotalus atrox venom then is added, and the incubation is continued for 3 more minutes (15 minutes total). The reaction is stopped by addition of 200 mL of activated charcoal (25 mg/-mL suspension in 0.1 M NaH₂PO₄, pH 4). After centrafugation (750×g for 3 minutes) to sediment the charcoal, a sample of the supernatant is taken for radioactivity determination in a scintillation counter and the PDE5 activity is calculated. The preparations had specific activities of about 3 μmoles cGMP hydrolyzed per minute per milligram protein.

### Bovine PDE6 Preparation

Bovine PDE6 was supplied by Dr N. Virmaux, INSERM U338, Strasbourg. Bovine retinas were prepared as described by Virmaux et al., FEBS Letters, 12(6), pp. 325-328 (1971) and see also, A. Sitaramayya et al., Exp. Eye Res., 25, pp. 163-169 (1977). Briefly, unless stated otherwise, all operations were done in the cold and in dim red light. Eyes were kept in the cold and in the dark for up to four hours after slaughtering.

Preparation of bovine retinal outer segment (ROS) basically followed procedures described by Schichi et al., J. Biol. Chem., 224:529 (1969). In a typical experiment, 35 bovine retinas were ground in a mortar with 35 mL 0.066 M phosphate buffer, pH 7.0, made up to 40% with sucrose, followed by homogenization in a Potter homogenizer (20 up and down strokes). The suspension was centrifuged at 25,000×g for 20 minutes. The pellet was homogenized in 7.5 mL 0.006 M phosphate buffer (40% in sucrose), and carefully layered under 7.5 mL of phosphate buffer (containing no sucrose). Centrifugation was conducted in a swing-out rotor at 45,000×g for 20 minuzes, and produced a pellet which is black at the bottom, and also a red band at the interface 0.066 M. phosphate—40% sucrose/0.066 M phosphate (crude ROS). The red material at the interface was removed, diluted with phosphate buffer, spun down to a pellet, and redistributed in buffered 40% sucrose as described above. This procedure was repeated 2 or 3 times until no pellet was formed. The purified ROS was washed in phosphate buffer and finally spun down to a pellet at 25,000×g for 20 minutes. All materials were then kept frozen until used.

Hypotonic extracts were prepared by suspending isolated ROS in 10 mM Tris-Cl pH 7.5, 1 mM EDTA, and 1 mM dithioerythritol, followed by centrifugation at 100,000×g for 30 minutes.

The preparation was reported to have a specific activity of about 35 nmoles cGMP hydrolyzed per minute per milligram protein.

### PDE1c Preparation from Spodoptera fugiperda Cells (Sf9)

Cell pellets (5 g) were thawed on ice with 20 ml of Lysis Buffer (50 mM MOPS pH 7.4, 10 μM ZnSO₄, 0.1 mM CaCl₂, 1 mM DTT, 2 mM benzamidine HCl, 5 μg/ml each of pepstatin, leupeptin, and aprotenin). Cells were lysed by passage through a French pressure cell (SLM-Aminco) while temperatures were maintained below 10° C. The resultant cell homogenate was centrifuged at 36,000 rpm at 4° C. for 45 minutes in a Beckman ultracentrifuge using a Type TI45 rotor. The supernatant was discarded and the resultant pellet was resuspended with 40 ml of Solubilization Buffer (Lysis Buffer containing 1M NaCl, 0.1M MgCl₂, 1 mM CaCl₂, 20μg/ml calmodulin, and 1% Sulfobetaine SB12 (Z3-12) by sonicating using a VibraCell tuner with a microtip for 3×30 seconds. This was performed in a crushed ice/salt mix for cooling. Following sonication, the mixture was slowly mixed for 30 minutes at 4° C. to finish solubilizing membrane bound proteins. This mixture was centrifuged in a Beckman ultracentrifuge using a type TI45 rotor at 36,000 rpm for 45 minutes. The supernatant was diluted with Lysis Buffer containing 10 μg/ml calpain inhibitor I and II. The precipitated protein was centrifuged for 20 minutes at 9,000 rpm in a Beckman JA-10 rotor. The recovered supernatant then was subjected to Mimetic Blue AP Agarose Chromatography.

In order to run the Mimetic Blue AP Agarose Column, the resin initially was shielded by the application of 10 bed volumes of 1% polyvinylpyrrolidine (i.e., MW of 40,000) to block nonspecific binding sites. The loosely bound PVP-40 was removed by washing with 10 bed volumes of 2M NaCl, and 10 mM sodium citrate pH 3.4. Just prior to addition of the solubilized PDE1c sample, the column was equilibrated with 5 bed volumes of Column Buffer A (50 mM MOPS pH 7.4, 10 μM ZnSO₄, 5 mM MgCl₂, 0.1 mM CaCl₂, 1 mM DTT, 2 mM benzamidine HCl).

The solubilized sample was applied to the column at a flow rate of 2 ml/min with recycling such that the total sample was applied 4 to 5 times in 12 hours. After loading was completed, the column was washed with 10 column volumes of Column Buffer A, followed by 5 column volumes of Column Buffer B (Column Buffer A containing 20 mM 51′-AMP), and followed by 5 column volumes of Column Buffer C (50 mM MOPS pH 7.4, 10 μM ZnSO₄, 0.1 mM CaCl₂, 1 mM dithiothreitol, and 2 mM benzamidine HCl). The enzyme was eluted into three successive pools. The first pool consisted of enzyme from a 5 bed volume wash with Column Buffer C. containing 1 mM cAMP. The second pool consisted of enzyme from a 10 bed volume wash with Column Buffer C containing 1 M NaCl. The final pool of enzyme consisted of a 5 bed volume wash with Column Buffer C containing 1 M NaCl and 20 mM cAMP.

The active pools of enzyme were collected and the cyclic nucleotide removed via conventional gel filtration chromatography or chromatography on hydroxy-apatite resins. Following removal of cyclic nucleotides, the enzyme pools were dialyzed against Dialysis Buffer containing 25 mM MOPS pH 7.4, 10 μM ZnSO₄, 500 mM NaCl, 1 mM CaCl₂, 1 mM dithiothreitol, 1 mM benzamidine HCl, followed by dialysis against Dialysis buffer containing 50% glycerol. The enzyme was quick frozen with the aid of dry ice and stored at −70° C.

The resultant preparations were about >90% pure by SDS-PAGE. These preparations had specific activities of about 0.1 to 1.0 μmol cAMP hydrolyzed per minute per milligram protein.

### IC₅₀ Value Determinations

The parameter of interest in evaluating the potency of a competitive enzyme inhibitor of PDE5 and/or PDE1c and PDE6 is the inhibition constant, i.e., Kᵢ. This parameter can be approximated by determining the IC₅₀, which is the inhibitor concentration that results in 50% enzyme inhibition, in a single dose-response experiment under the following conditions.

The concentration of inhibitor is always much greater than the concentration of enzyme, so that free inhibitor concentration (which is unknown) is approximated by total inhibitor concentration (which is known).

A suitable range of inhibitor concentrations is chosen (i.e., inhibitor concentrations at least several fold greater and several fold less than the Kᵢ are present in the experiment). Typically, inhibitor concentrations ranged from 10 nM to 10 μM.

The concentrations of enzyme and substrate are chosen such that less than 20% of the substrate is consumed in the absence of inhibitor (providing, e.g., maximum substrate hydrolysis of from 10 to 15%), so that enzyme activity is approximately constant throughout the assay.

The concentration of substrate is less than one-tenth the Michaelis constant (Kₘ). Under these conditions, the IC₅₀ will closely approximate the Kᵢ. This is because of the Chena-Prusoff equation relating these two parameters: IC₅₀=Kᵢ(1+S/Kₘ), with (1+S/Kₘ) approximately 1 at low values of S/Kₘ.

The IC₅₀ value is estimated from the data points by fitting the data to a suitable model of the enzyme inhibitor interaction. When this interaction is known to involve simple competition of the inhibitor with the substrate, a two-parameter model can be used:

Y=A/(1+x/B)

where the y is the enzyme activity measured at an inhibitor concentration of x, A is the activity in the absence of inhibitor and B is the IC₅₀. See Y. Cheng et al., Biochem. Pharmacol., 22:3099-3108 (1973).

Effects of inhibitors of the present invention on enzymatic activity of PDE5 and PDE6 preparations as described above were assessed in either of two assays which differed from each other principally on the basis of scale and provided essentially the same results in terms of IC₅₀ values. Both assays involved modification of the procedure of Wells et al., Biochim. Biophys. Acta, 384:430 (1975). The first of the assays was performed in a total volume of 200 μl containing 50 mM Tris pH 7.5, 3 mM Mg acetate, 1 mM EDTA, 50 μg/mL snake venom nucleotidase and 50 nM [³H]-cGMP (Amersham). Compounds of the invention were dissolved in DMSO finally present at 2% in the assay. The assays were incubated for 30 minutes at 30° C. and stopped by addition of 800 μl of 10 mM Tris pH 7.5, 10 mM EDTA, 10 mM theophylline, 0.1 mM adenosine, and 0.1 mM guanosine. The mixtures were loaded on to 0.5 mL QAE Sephadex columns, and eluted with 2 mL of 0.1 M formate (pH 7.4). The eluted radioactivity was measured by scintillation counting in Optiphase Hisafe 3.

A second, microplate, PDE assay was developed using Multiscreen plates and a vacuum manifold. The assay (100 μl) contained 50 mM Tris pH 7.5, 5 mM Mg acetate, 1 mM EDTA and 250 μg/mL snake venom nucleotidase. The other components of the reaction mixture were as described above. At the end of the incubation, the total volume of the assays were loaded on a QAE Sephadex microcolumn plate by filtration. Free radioactivity was eluted with 200 μl of water from which 50 μl aliquots were analyzed by scintillation counting as described above.

The following examples are presented to further illustrate the preparation of the claimed invention. The scope of the present invention is not to be construed as merely consisting of the following examples.

### EXAMPLE 1

The compound of structural formula (I) was prepared as described in U.S. Pat. No. 5,859,006 and formulated in tablets using wet granulation. Povidone was dissolved in water to make a 10% solution. The active compound, microcrystalline cellulose, croscarmellose sodium, and sodium lauryl sulfate were added to a high shear mixer and mixed for 2 minutes. The powders were wet granulated with the povidone solution and extra water as required to complete the granulation. The resultant mixture was dried in a fluid bed drier with inlet air at 70° C.±5° C. until the loss on drying was below 2.5%. The granules were passed through a Comil with a suitable screen (or a sieve) and added to a suitable mixer. The extragranular croscarmellose sodium arid sodium lauryl sulfate, and the colloidal anhydrous silica were passed through a suitable sieve (e.g., 500 micron) and added to the mixer and blended 5 minutes. Magnesium stearate was added and blended for 2 minutes. The blend was compressed to a target compression/weight of 250 mg using 9 mm round normal concave tooling.

The core tablets were coated with an aqueous suspension of Opadry OY-S-7322 using an Accelacota (or similar coating pan) using inlet air at 50° C. to 70° C. until the tablet weight was increased by approximately 8 mg. Opadry OY-S-7322 contains methylhydroxypropylcellulose Ph.Eur., titanium dioxide Ph. Eur., Triacetin USP. Opadry increases the weight of each tablet to about 258 mg. The amount of film coat applied per tablet may be less than that stated depending on the process efficiency.

The tablets are filled into blister packs and accompanied by package insert describing the safety and efficacy of the compound.

| | |
|---|---|
| | Formulations |
| Component | (mg per tablet) |

| | | |
|---|---|---|
| Selective PDE5 Inhibitor¹⁾ | 1 | 5 |
| Hydroxypropylmethylcellulose | 1 | 5 |
| phthalate | | |
| Microcrystalline Cellulose | 221.87 | 213.87 |
| Croscarmellose Sodium | 5.00 | 5.00 |
| Sodium Lauryl Sulfate | 2.50 | 2.50 |
| Sulfate Povidone K30 | 9.38 | 9.38 |
| Purified Water, USP (water | q.s. | q.s. |
| for irrigation) | | |
| Croscarmellose Sodium | 5.00 | 5.00 |
| Sodium Lauryl Sulfate | 2.50 | 2.50 |
| Colloidal Anhydrous Silica | 0.50 | 0.50 |
| Magnesium Stearate | 1.25 | 1.25 |
| Total core subtotal | 250.00 | 250.00 |
| (Film coat Opadry OY-S-7322) | about 8 mg | about 8 mg |

¹⁾Compound of structural formula (I).

### EXAMPLE 2

The following formula is used in preparing a finished dosage form containing 10 mg of the compound of structural formula (I).

| Ingredient | Quantity (mg) |
|---|---|
| Granulation | |
| Selective PDE5 Inhibitor¹⁾ | 10.00 |
| Lactose Monohydrate | 153.80 |
| Lactose Monohydrate (spray dried) | 25.00 |
| Hydroxypropyl cellulose | 4.00 |
| Croscarmellose Sodium | 9.00 |
| Hydroxypropylcellulose (EF) | 1.75 |
| Sodium Lauryl Sulfate | 0.70 |
| | 35.00 |
| Outside Powders | |
| Microcrystalline Cellulose (granular-102) | 37.50 |
| Croscarmellose Sodium | 7.00 |
| Magnesium Stearate (vegetable) | 1.25 |
| | Total 250 mg |
| Film coat (approximately) | 11.25 |

Purified Water, USP is used in the manufacture of the tablets. The water is removed during processing and minimal levels remain in the finished product.

Tablets are manufactured using a wet granulation process. A step-by-step description of the process is as follows. The drug and excipients to be granulated are security sieved. The selective PDE5 inhibitor is dry blended with lactose monohydrate (spray dried), hydroxypropylcellulose, croscarmellulose sodium, and lactose monohydrate. The resulting powder blend is granulated with an aqueous solution of hydroxypropylcellulose and sodium lauryl sulfate using a Powrex or other suitable high shear granulator. Additional water can be added to reach the desired endpoint. A mill can be used to delump the wet granulation and facilitate drying. The wet granulation is dried using either a fluid bed dryer or a drying oven. Once the material is dried, it can be sized to eliminate any large agglomerates. Microcrystalline cellulose, croscarmellose sodium, and magnesium stearate are security sieved and added to the dry sized granules. These excipients and the dry granulation are mixed until uniform using a tumble bin, ribbon mixer, or other suitable mixing equipment. The mixing process can be separated into two phases. The microcrystalline cellulose, croscarmellose sodium, and the dried granulation are added to the mixer and blended during the first phase, followed by the addition of the magnesium stearate to this granulation and a second mixing phase.

The mixed granulation then is compressed into tablets using a rotary compression machine. The core tablets are film coated with an aqueous suspension of the appropriate color mixture in a coating pan (e.g., Accela Cota). The coated tablets can be lightly dusted with talc to improve tablet handling characteristics.

The tablets are filled into plastic containers (30 tablets/container) and accompanied by package insert describing the safety and efficacy of the compound.

### EXAMPLE 3

The following formula is used -n preparing a finished dosage form of 5 mg of the compound of structural formula (I).

| Ingredient | Quantity (mg) |
|---|---|
| Granulation | |
| Selective PDE5 Inhibitor¹⁾ | 2.50 |
| Lactose Monohydrate | 79.395 |
| Lactose Monohydrate (spray dried) | 12.50 |
| Hydroxypropylcellulose | 2.00 |
| Croscarmellose Sodium | 4.50 |
| Hydroxypropylcellulose (EF) | 0.875 |
| Sodium Lauryl Sulfate | 0.35 |
| Outside Powders | |
| Microcrystalline Cellulose (granular- | 18.75 |
| 102) | |
| Croscarmellose Sodium | 3.50 |
| Magnesium Stearate (vegetable) | 0.63 |
| | Total 125 mg |
| Film coat (approximately) | 6.875 |

The dosage form of Example 3 was prepared in an identical manner to the dosage form of Example 2.

### EXAMPLE 4

Solution Capsule

| Ingredient | mg/Capsule | Percent (%) |
|---|---|---|
| Selective PDE5 Inhibitor^{1.} | 10 | 2 |
| PEG400 NF | 490 | 98 |
| Fill weight | 500 | 100 |

The gelatin capsules are precisely filled by pumping an accurate fill volume of predissolved drug formulation into the partially sealed cavity of a capsule. Immediately following injection fill of the drug solution formulation, the capsule is completely heat sealed.

The capsules are filled into plastic containers and accompanied by a package insert.

### EXAMPLE 5

In two randomized, double-blinded placebo controlled studies, the compound of structural formula (I), at a range of doses in both daily dosing and for on demand therapy for sexual encounters and intercourse in the home setting, was administered to patients in need thereof. Doses from 5 to 20 mg of the compound of structural formula (I) were efficacious and demonstrated no flushing and no reports of vision abnormalities. It was found that a 10 mg dose of the compound of structural formula (I) was fully efficacious and demonstrated minimal side effects (no flushing and no reports of blue vision).

Erectile function was assessed by the International Index of Erectile Function (IIEF) (Rosen et al., Urology, 49, pp. 822-830 (1997,), diaries of sexual attempts, and a global satisfaction question. The compound of structural formula (I) significantly improved erectile function as assessed by all end-points. In both “on demand” and daily dose regimens, the compound of structural formula (I) significantly improved erectile function in doses between 1 and 20 mg.

### EXAMPLE 6

Data from five clinical studies were integrated to show the efficacy of daily dosing of 5 mg and 10 mg of a compound of structural formula (I) (Study Drug). One study was of eight weeks duration, and the other four studies were of twelve weeks duration. The Study Drug was administered “daily” to patients with male erectile dysfunction. “Erectile dysfunction (ED)” is defined as the persistent inability to attain and/or maintain an erection adequate to permit satisfactory sexual performance.

The study population consisted of four subgroups as follows: (a) Study Drug taken less than 30% of the time during the study; (b) Study Drug taken 30% to 50% of the time during the study; (c) Study Drug taken 50% to 70% of the time during the study; and (d) Study Drug taken greater than 70% of the time during the study.

The Study Drug was orally administered as tablets of coprecipitate of Study Drug made in accordance with Butler U.S. Pat. No. 5,985,326 and as tablets containing the Study Drug as a free drug. The Study Drug was administered in 5 mg and 10 mg doses, “daily” and not more than once every 24 hours. No other approved or experimental medications, treatments, or devices used to treat ED were allowed.

The two primary efficacy variables were the ability of a subject to penetrate his partner and his ability to maintain an erection during intercourse, as measured by the International Index of Erectile Function (IIEF). The IIEF Questionnaire contains fifteen questions, and is a brief, reliable measure of erectile function. See R. C. Rosen et al., Urology, 49, pp. 822-830 (1997).

Secondary efficacy variables were IIEF domain scores for erectile function, orgasmic function, sexual desire, intercourse satisfaction, and overall satisfaction; the patient's ability to achieve an erection, ability to insert his penis into his partner's vagina, completion of intercourse with ejaculation, satisfaction with the hardness of his erection, and overall satisfaction, all as measured by the Sexual Encounter Profile (SEP) diary, especially, Question 2 and Question 3. The SEP is a patient diary instrument documenting each sexual encounter during the course of the study.

The safety analysis of the study included all enrolled subjects, and was assessed by evaluating all reported adverse events, and changes in clinical laboratory values, vital signs, physical examination results, and electrocardiogram results.

Overall, integration of the five studies demonstrated a trend toward better response with increased frequency of dose, both in the 5 mg and 10 mg group, and in all three primary efficacy variables. The results are summarized in following Tables 2-4.

TABLE 2
Summary of IIEF Erectile Function Domain

| | | Percent of the time taken drug during | | | |
|---|---|---|---|---|---|
| | | the study | | | |
| Dose | Statistics | <30% | 30% to 50% | 50% to 70% | >70% |
| 5 mg | N | 97 | 54 | 28 | 13 |
| | Mean Baseline | 13.2 | 13.5 | 14.1 | 13.1 |
| | Mean Endpoint | 17.4 | 17.5 | 20.9 | 22.1 |
| | Mean change | 4.3 | 4.0 | 6.8 | 9.0 |
| 10 mg | N | 164 | 75 | 41 | 43 |
| | Mean Baseline | 14.2 | 14.4 | 13.9 | 14.8 |
| | Mean Endpoint | 20.0 | 21.4 | 21.5 | 22.2 |
| | Mean Change | 5.9 | 6.9 | 7.6 | 7.4 |

TABLE 3
Summary of SEP Question 2
(Ability to insert penis)

| | | Percent of the time taken drug during | | | |
|---|---|---|---|---|---|
| | | the study | | | |
| Dose | Statistics | <30% | 30% to 50% | 50% to 70% | >70% |
| 5 mg | N | 98 | 54 | 28 | 13 |
| | Mean Baseline | 42.7 | 40.8 | 47.9 | 42.8 |
| | Mean Endpoint | 57.2 | 57.2 | 69.3 | 68.2 |
| | Mean change | 14.4 | 16.5 | 21.4 | 25.5 |
| 10 mg | N | 164 | 76 | 41 | 45 |
| | Mean Baseline | 44.7 | 47.5 | 436 | 45.9 |
| | Mean Endpoint | 66.2 | 69.0 | 73.4 | 75.6 |
| | Mean Change | 21.5 | 21.5 | 29.9 | 29.7 |

TABLE 4
Summary of SEP Question 3
(Sufficiently long erection for successful intercourse)

| | | Percent of the time taken drug during | | | |
|---|---|---|---|---|---|
| | | the study | | | |
| Dose | Statistics | <30% | 30% to 50% | 50% to 70% | >70% |
| 5 mg | N | 98 | 54 | 28 | 13 |
| | Mean Baseline | 21.8 | 16.7 | 18.7 | 18.4 |
| | Mean Endpoint | 38.2 | 40.4 | 53.5 | 54.6 |
| | Mean Change | 16.4 | 23.7 | 33.8 | 36.2 |
| 10 mg | N | 164 | 76 | 41 | 45 |
| | Mean Baseline | 24.6 | 26.5 | 20.2 | 25.3 |
| | Mean Endpoint | 53.5 | 56.3 | 63.2 | 63.9 |
| | Mean Change | 28.9 | 29.7 | 43.0 | 38.6 |

### EXAMPLE 7

A double-blind, placebo-controlled study assessed the safety and efficacy of daily treatment using a compound of formula (I) (Study Drug) in men 21-72 years of age and experiencing mild to moderate erectile dysfunction. Men having a history of radical prostatectomy or diabetes mellitus were excluded In this study, following a three-week treatment free run-in period, the subjects were randomized to a three week daily treatment with placebo or Study Drug (10, 25, 50, or to 100 mg). All participants in the study agreed to attempt four sexual encounters during both the run-in and treatment periods. Baseline International Index of Erectile Function (IIEF) scores, sexual encounter profile (SEP) diary data, and the global assessment question (GAQ) were collected during the treatment period. Primary endpoints were change from baseline in Questions 3 (treatment effect on penetration ability) and 4 (treatment effect on erection maintenance) of the IIEF. Secondary endpoints included change from baseline in all IIEF domains and in SEP and GAQ responses. The results for the group administered 10 mg of Study Drug daily were comparable to, or better than, results for groups administered 25, 50, and 100 mg of Study Drug daily.

Compared to the placebo, the Study Drug significantly improved erectile function as assessed by all study endpoints. For example, in groups treated with the Study Drug, the change in IIEF Question 3 was about 1.4 (compared to placebo) with daily 10 mg treatment The change in Question 4 was about 1.8 (compound to placebo) with 10 mg daily treatment. Successful intercourse rates using the Study Drug, as reported in SEP diaries, were up to 82% with 10 mg daily therapy, compared to 40.4% for placebo. In addition, 90% of the subject receiving 10 mg daily dose of Study Drug reported improved erection on the GAQ compared to 30% of subjects administered a placebo. Adverse events were dose-related, and attenuated with continued daily treatment. The most common adverse events were headache, back pain, myalgia, and dyspepsia. Treatment-related headache, the most common adverse event, was observed in 13% to 46% of subjects receiving daily Study Drug compared to 3% for placebo. There were no treatment-related changes in vital signs, ECG, or laboratory measures.

In accordance with the present invention, a daily unit dose of about 1 to about 10 mg, preferably about 2 to about 10 mg, and most preferably about 5 to about 10 mg, administered daily up to a maximum of 10 mg per day for at least three days, effectively treats ED, minimizes or eliminates the occurrence of adverse side effects, and improves vascular conditioning. Importantly, the patient is provided spontaneity with respect to sexual activities and a more rapid return to a prearoused state. Surprisingly, in addition to treating ED in individuals, a greater response was observed using a low daily dose compared to a higher on-demand dose of PDE5 inhibitor, in addition to a lower instances of adverse events attributed to lower dose.

The principles, preferred embodiments, and modes of operation of the present invention have been described in the foregoing specification. The invention intended to be protected herein, however, is not construed to be limited to the particular forms disclosed, because they are to be regarded as illustrative rather than restrictive. Variations and changes may be made by those skilled in the art without departing from the spirit of the invention.

What is claimed is:

1. An article of manufacture for human pharmaceutical use comprising:
(a) an oral dosage form comprising a PDE5 inhibitor having an IC₅₀ for the inhibition of PDE5 less than 10 nM, and sufficient bioavailability to be effective in about 1 to about 10 mg unit oral dosages;
(b) a package insert providing that the PDE5 inhibitor is useful to treat sexual dysfunction in a patient in need thereof by utilizing a chronic dosing regimen; and
(c) a container.

2. An article of manufacture for human pharmaceutical use comprising:
(a) an oral dosage form comprising a PDE5 inhibitor having an IC₅₀ less than 10 nM, and a sufficient bioavailability to be effective in about 1 to about 10 mg unit oral dosages;
(b) a package insert providing that the PDE5 inhibitor is useful to treat sexual dysfunction in a patient in need thereof by utilizing a chronic dosing regimen, wherein the chronic dosing regimen improves vascular conditioning; and
(c) a container.

3. An article of manufacture for human pharmaceutical use comprising:
(a) an oral dosage form comprising a PDE5 inhibitor having an IC₅₀ less than 10 nM, and a sufficient bioavailability to be effective in about 1 to about 10 mg unit oral dosages;
(b) a package insert providing that the PDE5 inhibitor is useful to treat sexual dysfunction in a patient in need thereof by utilizing a chronic dosing regimen, wherein the chronic dosing regimen improves vascular conditioning compared to an acute or on-demand dosing of sildenafil; and
(c) a container.

4. An article of manufacture for human pharmaceutical use comprising:
(a) an oral dosage form comprising a PDE5 inhibitor having an IC₅₀ less than 10 nM, and a sufficient bioavailability to be effective in about 1 to about 10 mg unit oral dosages;
(b) a package insert providing that the PDE5 inhibitor is useful to treat sexual dysfunction in a patient in need thereof by utilizng a chronic dosing regimen, wherein the chronic dosing regimen improves vascular conditioning compared to an acute or on-demand dosing of vardenafil; and
(c) a container.

5. The article of manufacture of claims 1 through 4, wherein the PDE5 inhibitor further has
(i) at least a 100 fold differential in IC₅₀ values for the inhibition of PDE5 versus PDE6, and
(ii) at least 1000 fold differential in IC₅₀ values for the inhibition of PDE5 versus PDE1c.

6. The article of claims 1 through 4 wherein the oral dosage form comprises about 1 mg, about 2 mg, about 5 mg, or about 10 mg, of the PDE5 inhibitor.

7. The article of claims 1 through 4 wherein the chronic dosing regimen is a daily dosing regimen.

8. The article of claims 1 through 4 wherein the chronic dosing regimen comprises administration of about 1 mg/day to about 10 mg/day of the PDE5 inhibitor.

9. The article of claims 1 through 4 wherein the package insert provides a maximum dosage of the PDE5 inhibitor of about 10 mg per day.

10. The article of claims 1 through 4 wherein the PDE5 inhibitor is selected from the group consisting of
(6R, 12aR)-2,3,6,7,12,12a-hexahydro-2-methyl-6-(3,4-methylenedioxyphenyl)pyrazino[2′,1′:6,]pyrido[3,4-b]indole-1,4-dione;
(3S,6R,12aR)-2,3,6,7,12,12a-hexahydro-2,3-dimethyl-6-(3,4-methylenedioxyphenyl)pyrazino[2′,1′:6,1]-pyrido[3,4-b]indole-1,4-dione;
5-(2-ethoxy-5-morpholinoacetylphenyl)-1-methyl-3-n-propyl-1,6-dihydro-7H-pyrazolo[4,3-d]pyrimidin-7-one;
5-(5-morpholinoacetyl-2-n-propoxyphenyl)-1-methyl-3-n-propyl-1,6-dihydro-7H-pyrazolo[4,3-d]pyrimidin-7-one;
5-[2-allyloxy-5-(4-methyl-1-piperazinylsulphonyl)-phenyl]-1-methyl-3-n-propyl-1,6-dihydro-7H-pyrazolo-[4,3-d]pyrimidin-7-one;
5-{2-ethoxy-5-[4-(2-propyl)-1-piperazinylsulphonyl]-phenyl}-1-methyl-3-n-propyl-1,6-dihydro-7H-pyrazolo-[4,3-d]pyrimidin-7-one;
5-{2-ethoxy-5-[4-(2-hydroxyethyl)-1-piperazinylsulphonyl)phenyl }-1-methyl-3-n-propyl-1,6-dihydro-7H-pyrazolo[4,3-d]pyrimidin-7-one;
5-{5-[4-(2-hydroxyethyl)-1-piperazinylsulphonyl]-2-n-propoxyphenyl}-1-methyl-3-n-propyl-1,6-dihydro-7H-pyrazolo[4,3-d]pyrimidin-7-one;
5-[2-ethoxy-5-(4-methyl-1-piperazinylcarbonyl)-phenyl]-1-methyl-3-n-propyl-1,6-dihydro-7H-pyrazolo-[4,3-d]pyrimidin-7-one; and
5-[2-ethoxy-5-(1-methyl-2-imidazolyl)phenyl]-1-methyl-3-n-propyl-1,6-dihydro-7H-pyrazolo[4,3-d]pyrimidin-7-one.

11. The article of claim 10 wherein the chronic dosing regimen comprises administration of about 1 mg/day to about 10 mg/day cf the PDE5 inhibitor.

12. The article of claims 1 through 4 wherein the PDE5 inhibitor is selected from the group consisting of sildenafil and vardenafil.

13. The article of claims 1 through 4, wherein the PDE5 inhibitor has the structure

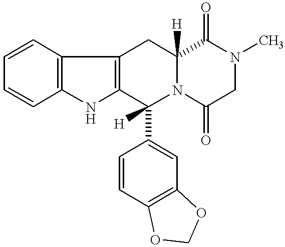

14. A method of treating sexual dysfunction comprising using an article of manufacture of claims 1 through 4.

15. A method of treating sexual dysfunction comprising a chronic administration to an individual in need thereof of one or more oral dosage form of a PDE5 inhibitor in an amount of about 1 mg/day to about 10 mg/day for at least three days.

16. The method of claim 15 wherein the chronic administration of a PDE5 inhibitor is a daily administration.

17. A method of improving a relaxant response in corpus cavernosum smooth muscle comprising a chronic administration of a PDE5 inhibitor selected from (6R,12aR)-2,3,6,7,12,12a-hexahydro-2-methyl-6-(3,4-methylenedioxyphenyl)pyrazino[2′,1′:6,1]-pyrido[3,4-b]indole-1,4-dione for at least three days.

18. The method of claim 17 comprising the chronic administration of about 1 mg/day to about 10 mg/day of the PDE5 inhibitor.
